(19) United States
(12) Patent Application Publication
(10) Pub. No.: US 2022/0296857 A1
(43) Pub. Date: Sep. 22, 2022

(54) SECUREMENT DEVICE AND SECUREMENT KIT

(71) Applicant: GRIFFITH UNIVERSITY, Red Hill, Brisbane, Queensland (AU)

(72) Inventors: Andrew BULMER, Red Hill, Brisbane, Queensland (AU); Derek SMITH, Red Hill, Brisbane, Queensland (AU); Timothy SCOTT, Red Hill, Brisbane, Queensland (AU)

(73) Assignee: GRIFFITH UNIVERSITY, Red Hill, Brisbane, Queensland (AU)

(21) Appl. No.: 17/612,955

(22) PCT Filed: May 20, 2020
(86) PCT No.: PCT/AU2020/050493

§ 371 Date: Nov. 19, 2021

(30) Foreign Application Priority Data
May 20, 2019 (AU) 2019901705

(51) Int. Cl. A61M 25/02; A61M 25/00

(52) U.S. Cl. CPC A61M 25/02; A61M 25/0043; A61M 2025/024; A61M 2025/0266; A61M 2025/028; A61M 2205/0205; A61M 2205/0238; A61M 2025/0056; A61M 2207/10

(57) ABSTRACT

A securement device for securement of a catheter connecting assembly to a body of a patient wherein the catheter connecting assembly includes two or more ports adapted to be coupled with the catheter, each port being formed by a respective port body wherein the ports are adapted to be fluidly coupled to a catheter hub of a catheter, the securement device comprising: a base having a continuous top surface and a continuous bottom surface wherein at least a portion of the top surface comprises an antimicrobial layer and wherein at least a portion of the bottom surface comprises an adhesive for attachment of the base to the body of the patient and wherein the base is dimensioned to prevent direct contact between said ports and body of the patient; and one or more retention formations located on the top surface of the base for receiving and retaining at least a portion of a housing comprising said one or more port bodies adapted to be coupled with a terminal end of the catheter.

### TECHNICAL FIELD

The present invention relates to a securement device and a securement system used for securement of a medical line to a patient.

### BACKGROUND

Any references to methods, apparatus or documents of the prior art are not to be taken as constituting any evidence or admission that they formed, or form part of the common general knowledge.

It is common to use an implanted catheter to repeatedly access the vascular system of a patient and perform repeated therapeutic medical activity. Such therapeutic activity can include the infusion of medication and fluids, the periodic sampling of blood, or the continuous withdrawal and return of blood for processing outside of the body of the patient. The catheters used in these activities are generally referred to as vascular access catheters.

Before the commencement of any therapeutic activity, the vascular access catheter is implanted into the body of the patient with the tip of the catheter residing at the location in the vascular system at which an intended therapeutic activity is appropriate. Typically, most of the length of an implanted vascular access catheter resides within blood vessels of the vascular system, extending from the tip of the catheter to a location in the vascular system at which the catheter, by traversing a puncture or incision formed through a wall of the blood vessel into which the catheter is disposed. The location at which this occurs is referred to as a venipuncture site.

Away from the venipuncture site, the implanted catheter extends through the subcutaneous tissue of the patient to emerge through the skin at a location that is referred to as the skin exit site. In various applications, the extracorporeal portion of an implanted catheter needs to be configured to permit selective coupling and uncoupling between the tubing and the medical equipment outside the body of the patient that are required for therapeutic activity. Accordingly, the end of vascular access catheters are configured to terminate in a catheter coupling hub that can be secured in fluid communication with such tubing and medical equipment, or can be capped, valved, or clamped closed between periods of actual use.

Often, it becomes desirable to maintain catheterization over an extended period of time during the treatment of a patient. In order to keep the catheter or other medical lines properly positioned for the duration of treatment, the catheter or medical line can be secured to the patient in a variety of ways. Most commonly, this involves taping the catheter or medical line to the patient.

Securing a catheter with tape upon the patient traditionally has certain drawbacks. The use of tape at the insertion site can retain dirt or other contaminant particles, potentially leading to infection of the patient. Tape also fails to limit catheter motion and, therefore, contributes to motion related complications like phlebitis, infiltration and catheter migration. Additionally, removal of taped dressings can itself cause undesired motion of the catheter upon the patient.

Adhesive clips have been available for many years and are widely used and compatible with practically all types of peripherally inserted central catheters (PICCs) on the market. These adhesive fixation devices are available in several shapes and sizes. One commonly known adhesive clip is the Stetlock®, which is a foam-backed adhesive pad with two clips and prongs that fit into holes either side of the PICC hub. This type of fixation device has been used for many years and is supplied as standard with most PICCs as a component of the PICC pack. Adhesive clips have been found to be reliable but can be difficult to replace during dressing change and have been linked with local skin reactions. Another drawback with such clips is that they are used for securing the catheter hub to the skin of the patient. However, any medical equipment which typically consists of catheter extension kits (also known as extension sets) is left unsecured and can come into contact with various surfaces. FIGS. 1A to 1D illustrate a central line (with three connectors), a central line with three ports/needleless connectors, a PICC line and peripheral intravenous cannula (PIVC) line that use an adhesive clip for securement of the catheter hub but fail to provide any suitable securement for the catheter extension sets. These extensions can include extension sets that extend from the catheter hub and usually have devices attached to them (such as needle-less connectors, three-way valves, injection ports or further extension tubing).

In view of the above, it is desirable to provide a securement device and an improved catheter connector assembly that addresses some of the problems of the prior art.

### SUMMARY OF INVENTION

In an aspect, the invention provides a securement device for securement of a catheter connecting assembly to a body of a patient wherein the catheter connecting assembly includes two or more ports adapted to be coupled with the catheter, each port being formed by a respective port body wherein the ports are adapted to be fluidly coupled to a catheter hub of a catheter, the securement device comprising:

a base having a continuous top surface and a continuous bottom surface wherein at least a portion of the top surface comprises an antimicrobial layer and wherein at least a portion of the bottom surface comprises an adhesive for attachment of the base to the body of the patient and wherein the base is dimensioned to prevent direct contact between said ports and body of the patient; and

one or more retention formations located on the top surface of the base for receiving and retaining at least a portion of a housing comprising said one or more port bodies adapted to be coupled with a terminal end of the catheter.

In an embodiment, the retention formations are structured to prevent direct contact with the top surface of the base and the port bodies.

In an embodiment, said retention formations are located on said portion of the top surface comprising the antimicrobial layer.

In an embodiment, the base and the retention formations are integrally formed.

In an embodiment, the retention formations project in an in-use upwardly direction to receive and retaining at least a portion of a housing defining said one or more port bodies.

In an embodiment, the antimicrobial layer on the top surface of the base is an assembled film or a substrate coated with one or more antimicrobial additives.

In an embodiment, the device further comprises a removable covering to cover said portions of the top surface with the antimicrobial coating.

In an embodiment, the antimicrobial additives are antiseptic or disinfecting agents or agents that may repel pathogen adhesion or growth.

In an embodiment, the retention formations are structured to allow said at least portions of the housing defining the one or more port bodies to be press-fitted or snap-fitted thereon by way of pushing the housing in a generally downwardly direction into the top surface of the base.

In an embodiment, the retention formations comprise a plurality of clips, each clip being configured to allow a housing portion of the body to be snap fitted into the clip.

In an embodiment, each clip comprises a medial portion defining a receiving portion positioned in between biased arms that define an opening to receive the housing of the port body into the receiving portion.

In an embodiment, each of the biased arms includes an outwardly positioned guiding surface to direct the housing of a port body of the catheter connector towards the receiving portion.

In an embodiment, the medial portion of the clip member is adapted to receive and retain the housing of a port body to position the passage defined by the port body to extend in a substantially parallel direction relative to the top surface of the base.

In an embodiment, the retention formations define an opening to form a female connecting portion to receive a male connecting member positioned on the housing of the one or more port bodies of the catheter connector.

In an embodiment, each of the retention formations defining a substantially cylindrical body defining said female connecting portion, the cylindrical body projecting in an in-use upwardly direction from the top surface of the base so that the male connecting portion can be press-fitted into the female connecting portion.

In another aspect the invention is a securement assembly comprising a securement device as described herein in combination with the catheter connector.

In an embodiment, the male connecting member extends outwardly in a transverse direction relative to a longitudinal axis of the passageway defined by the port body.

In an embodiment, the male connecting member of the housing for the port body of the catheter connector and the female connecting portion of the retention formation are configured to position the longitudinal axis of the passageway defined by the port body in a substantially parallel direction relative to the top surface of the base.

In an embodiment, the retention formation comprises a male connecting portion extending upwardly from the top surface of the base, the male connecting portion being adapted for receiving a female connecting portion of the housing defining said one or more port bodies.

In an embodiment, the male connecting portion comprises a shoulder portion for engaging an end portion of the female connecting portion thereby allowing the female connecting portion to be press-fitted into the male connecting portion of the base.

In an embodiment, the housing further comprises a releasing member for uncoupling the female connecting portion from the male connecting portion of the securement device.

In an embodiment, the releasing member is movably positioned within a bore of the housing, the releasing member having an in-use upper end portion and an in-use lower end portion whereby pushing the releasing member in a generally downward direction results in the lower end portion of the releasing member being pushed against an upper portion of the male connecting portion.

In an embodiment, the securement assembly further comprises a biasing arrangement being arranged to apply a bias against the releasing member being pushed in said generally downward direction.

In an embodiment, the housing further comprises one or more inlet ports and one or more outlet ports, said inlet and outlet ports being provided along outer walls of the housing with a flow passage extending between said inlet and outlet ports in a transverse direction relative to the bore of the housing with a controller positioned relative to the housing for controlling flow of fluid from the inlet port to the outlet port.

In an embodiment, the release member comprises a cavity positioned between the upper end and the lower end of the release member, the cavity being dimensioned for accommodating the flow passage to be positioned therein during use.

In an embodiment, the release member comprises a constricted middle portion to define said cavity, the middle portion being dimensioned to be positioned between an inner wall of the housing and the walls of the flow passage extending across the internal volume of the housing.

In an embodiment, the biasing arrangement comprises a spring member.

In an embodiment, an in-use upper portion of the housing comprises a tab member for receiving a user's fingers to allow a user to simultaneously push down the releasing member in a downward direction and pull the housing in an upward direction to uncouple the female connecting portion of the housing from the male connecting portion of the securement device.

In an embodiment, the release member comprises a lever coupled to an engagement portion adapted to be positioned in between a lower portion of the housing and the male connecting portion of securement device such that movement of the lever in a first direction results in the female connecting portion of the housing becoming uncoupled from the securement device.

In an embodiment, the lever is coupled with a biasing member to apply a biasing force against movement of the lever in the first direction.

In an embodiment, the lever is arranged to pivot relative to an engagement portion located between a lower portion along an outer wall of the housing and the male connecting portion such that pivoting movement of the lever towards the outer walls of the housing results in the female connecting portion of the outer housing becoming uncoupled from the male connecting portion.

In an embodiment, the release member is movably located within an aperture positioned in an in-use lower portion of the housing wherein the release member comprises an inner portion such upon pushing the release member in an inwardly direction, the inner portion is wedged between the male connecting portion and the female connecting portion of the housing to separate the housing from the male connecting portion.

### BRIEF DESCRIPTION OF THE DRAWINGS

Preferred features, embodiments and variations of the invention may be discerned from the following Detailed Description which provides sufficient information for those skilled in the art to perform the invention. The Detailed Description is not to be regarded as limiting the scope of the preceding Summary of the Invention in any way. The Detailed Description will make reference to a number of drawings as follows:

FIGS. 1A to 1D show images of the prior art catheter connection assemblies coupled with a catheter hub.

FIG. 2A is a top view of a securement device 100 in accordance with an embodiment of the present invention.

FIG. 2B is a perspective view of the securement device 100.

FIG. 2C is an exploded frontal perspective view of the securement device 100.

FIG. 3A is a perspective view of a securement device 100A in accordance with another embodiment of the present invention.

FIG. 3B is a top view of the securement device 200.

FIG. 4A is an exploded perspective view of a securement device 200.

FIG. 4B is a side perspective view of the securement device 200.

FIG. 4C is another exploded perspective view of the securement device 200.

FIG. 4D is a side view of the securement device 200.

FIG. 4E is a rear view of the securement device 200.

FIG. 5A is a perspective view of a securement device 300 in combination with a stop cock assembly 360.

FIG. 5B is a sectional view of the securement device 300 in combination with a stop cock assembly 360.

FIG. 5C is a side view of the securement device 300 in combination with a stop cock assembly 360.

FIG. 5D is an exploded view of the securement device 300 in combination with a stop cock assembly 360.

FIG. 6A is a perspective view of the securement device 300 in combination with a stop cock assembly 460.

FIG. 6B is a perspective view of the securement device 300 in combination with the stop cock assembly 460.

FIG. 6C is a side internal view of the securement device 300 in combination with the stop cock assembly 460 (initial uncoupled configuration).

FIG. 6D is a side internal view of the securement device 300 in combination with the stop cock assembly 460 (coupled configuration).

FIG. 6E is a side internal view of the securement device 300 in combination with the stop cock assembly 460 (uncoupled configuration).

FIG. 7A is a first side view of the securement device 300 in combination with a stop cock assembly 560.

FIG. 7B is a second side view of the securement device 300 in combination with a stop cock assembly 560 (coupled configuration).

FIG. 7C is a side view of the securement device 300 in combination with a stop cock assembly 560 (uncoupled configuration).

FIG. 8A is a perspective view of the securement device 300 in combination with a stop cock assembly 660.

FIG. 8B is a first internal side view of the securement device 300 in combination with the stop cock assembly 660.

FIG. 8C is a second internal side view of the securement device 300 in combination with the stop cock assembly 660.

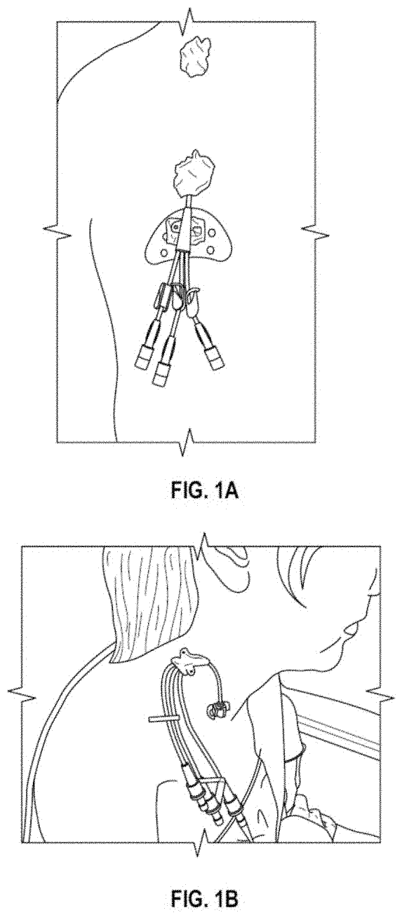

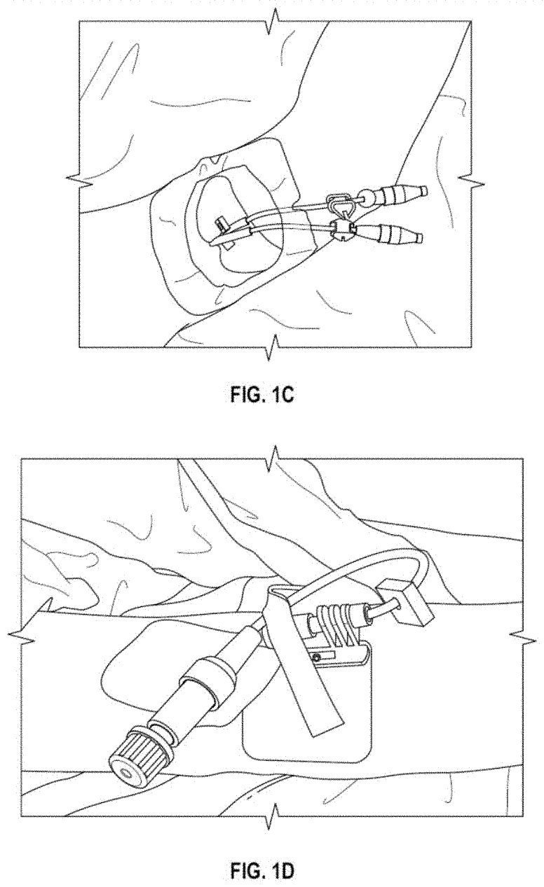

### DETAILED DESCRIPTION OF PREFERRED EMBODIMENTS

The following description and the accompanying figures, which describe and show the preferred embodiments, are made to demonstrate several possible configurations that a securement device or system may take to include various aspects and features the invention. The illustrated embodiments are shown in use with several types of connector fittings that are typically used in extension sets or medical equipment that is fluidly coupled with a catheter hub. The illustrative drawings do not show a catheter hub but instead show several types of connector fittings that may be secured with the securement device in accordance with several embodiments. The illustration of the securement device in this context is not intended to limit the disclosed aspects and features of the invention to the specified embodiments or to usage only with the illustrated connector or hub. Those of skill in the art will recognize that the disclosed aspects and features of the invention are not limited to any particular embodiment of a securement system, and securement systems, which include one or more of the inventive aspects and features herein described, can be designed for use with a variety of medical articles.

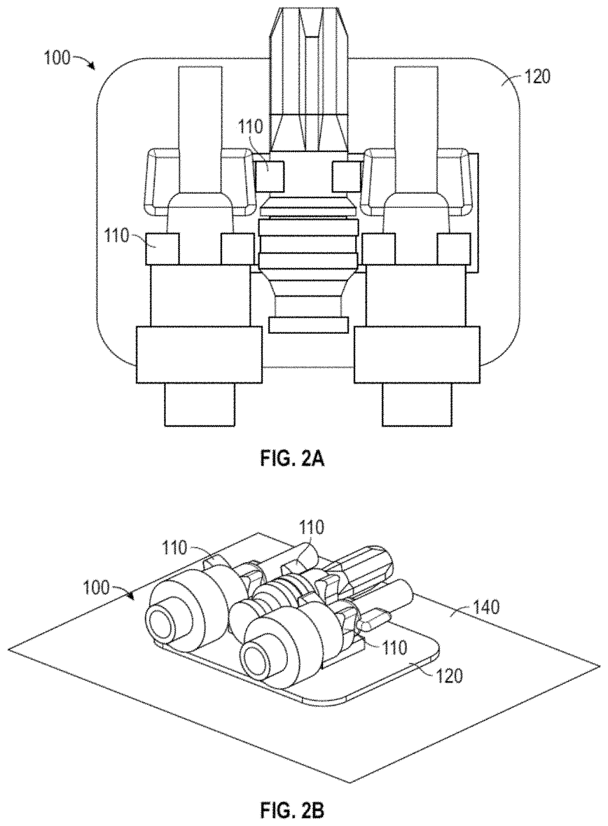

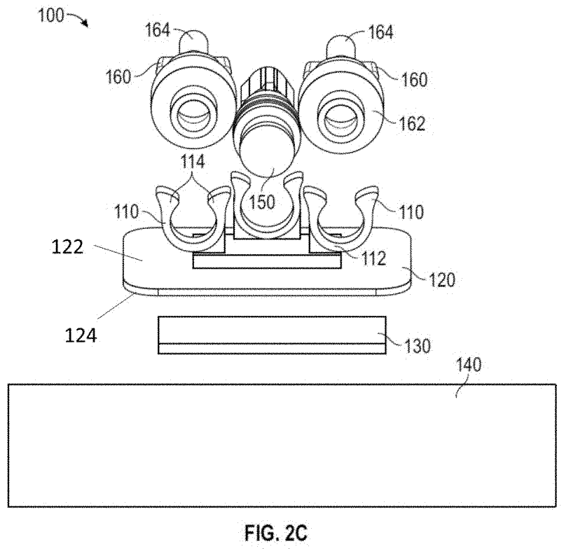

FIGS. 2A to 2C illustrate a securement device 100 in accordance with a first preferred embodiment of the present invention. The securement device 100 in the present embodiment is configured for securing a catheter connecting assembly that can be fluidly coupled to a catheter hub via fluid lines (not shown). The catheter connector assembly includes needless connectors comprising port bodies 150 and 160 that include an adaptor 162 for conveying infused fluid via the catheter hub by uncoupling the luer lock 164 and introducing fluids into the port bodies 160. Similarly, port body 150 may be used for sampling blood.

The securement device 100 comprises a base 120 having a continuous top surface 122 and an underside surface 124. The continuous top surface 122 may include an assembled film with a specified antimicrobial additive which may be exposed on the continuous top surface 122 in a manner such that it is away from the patient's skin. In one embodiment, the antimicrobial additive may be applied with an additive in the substrate base polymer, for example, polyurethane with antimicrobial additives. In another embodiment, the antimicrobial additive may be applied by coating a substrate to create an antimicrobial barrier layer. The coating process may involve one or more non-limiting methods wherein the antimicrobial additives may be applied through spraying, dipping, brushing or any other technique onto the continuous top surface 122. The base 120 including the underlying substrate receiving the antimicrobial coating may include PTFE (Polytetrafluoroethylene/Teflon) or other suitably biocompatible polymeric materials, which assist with surface binding of the antimicrobial additives. In alternative embodiments, surface treatments with organic anti-fouling agents such as polyethylene glycol (PEG) or superhydrophobic coatings with fluoroalkylated acrylic acid oligomers may also be used. In some embodiments, the antimicrobial additives may include Chlorhexidine (0.25-2.5% volume by weight) or other biocides, Silver nanoparticles at a tested biocompatible non-toxic concentration or other proven antimicrobial metallic elements such as Zinc or Copper nanoparticles. In other embodiments, only a single antimicrobial agent or surface treatment method may be applied depending upon the efficacy of the coatings in antimicrobial resistance.

The underside surface 124 also consists of an Adhesive patch of a two-part silicone base such as like DOW CORNING™ MG 7-1020 or other similar biocompatible adhesive material. An adhesive in the form of a chloroxylenol double sided polyethylene foam tape 130 is positioned on the underside surface of the base 120. In alternative embodiments, other medical-grade adhesives may also be used, depending upon the particular application. The securement device 100 comprises a retention formations in the form of retention clips 110 that are adapted to engage an outer wall of a housing that defines each of the port bodies 150 and 160 and to receive and retain the portion bodies 150 and 160 on the top surface of the base 110. Each of the clips 110 are integrally formed with the base 120 and project in an in-use upwardly direction with an upper portion of the projection defining an opening to receive the housing of each of the respective port bodies 150 and 160. Each clip member 110 comprises a curved medial portion 112 with biased end portions 114 that define an opening to receive the housing of respective port bodies 150 and 160. The end portions 114 included curved guiding surfaces. During use, the user may push the port bodies on these guiding surfaces which results in the end portions being spaced apart to accommodate the housing of the respective port body (150 or 160) there-between. The end portions 114 are biased towards each other and snap back to allow each of the port bodies 150 and 160 to be snap fitted into the clips 110. The use of the securement device 100 results in the base 120 being adhesively bonded to the patient's skin and securement of the port bodies 150 and 160 on the base 120.

It is important to note that portions of the top surface 122 below or adjacent the retention formations including the retention clips 110 or port bodies 150 and 160 include the antimicrobial coating as previously discussed. Although the invention as described is not be construed as relying upon any hypothesis as to mode of the action, it has been inferred by the inventors that locating the antimicrobial coating upon the top surface 122 of the base 120 at a location adjacent the retention formations such as but not limited to the retention clips 110 results in the antimicrobial material being released into the environment surrounding the port bodies 150 and 160 that are fluidly coupled with the terminal end of a catheter line. It has been found by the inventors that the terminal ends of the catheter lines that are coupled with the port bodies 150 and 160 are highly susceptible to bacterial infections due to the possible build-up on the top surface of any substrate that supports such port bodies. While it is standard practice to provide antibacterial coatings on the underside of such substrates, such practices have not been found to be as effective for mitigating catheter line infections. The inventors believe that forming the antimicrobial layer formed on the top surface at or adjacent the retention formations effectively inhibits bacterial adhesion and biofilm formations at and around locations surrounding the port bodies connected with the terminal end of the catheter lines. It is also desirable to provide the antimicrobial coating in the form of a water insoluble layer to prevent the antimicrobial coating from being inadvertently washed away upon exposure to water. In such instances, the antimicrobial layer might take the form of a hydrogel layer with antimicrobial metallic material such as but not limited to Silver (Ag) to release Ag⁺ ions in the vicinity of the port bodies 150 and 160. The inventors have hypothesized that the positioning of the antimicrobial additive layer on the continuous top surface 122 helps to prevent infection from developing inside or around the port bodies 150 and 160 which can then easily spread into the catheter line via the terminal end of the catheter line connected with the port bodies 150 and 160.

The provision of the antimicrobial agent on the top surface 122 of the base 120 may necessitate the inclusion of a removable or peelable covering for the coated portions of the top surface 122. As with most antibiotic agents, it is expected that the antimicrobial coating upon being exposed would have a limited shelf life ranging from a few hours up to a few days and even a few weeks. The provision of the removable covering would ensure that the user or the care giver peels off the removable covering once the securement device 100 is being used on a patient.

The novel arrangement of the securement device 100 also prevents the port bodies 150 and 160 and the connected fluid lines from being inadvertently caught or pulled whilst they are connected with the catheter hub. Moreover, the base 120 is coated with antiseptic and/or antimicrobial agent which further reduces chances of port access infection. Further improvement is also achieved by placing a chloroxylenol impregnated transparent film 140 which is placed over the user's body whilst also simultaneously bonding the base 120 to the transparent film. (by the adhesive tape 130). Such an arrangement of the port bodies 150 and 160 on top of the base 120 prevents direct contact between the port bodies 150 and 160 and the patient's skin thereby reducing the chances of bacterial infection. Without being bound by theory, the inventors hypothesize that the use of the additional antiseptic barrier in the form of the film 140 in combination with the securement device 100 can address the issue of central line-associated bloodstream infection (CLABSI) because the base 120 effectively provides a disinfecting barrier between the port bodies 150 and 160 (fastened and retained on the top surface of the base 120) and the body of the patient.

It is also important to note that in at least some embodiments, the invention may encompass a kit that includes the securement device 100 either alone or in combination with the port bodies 150 and 160 and other parts of the catheter extension assembly.

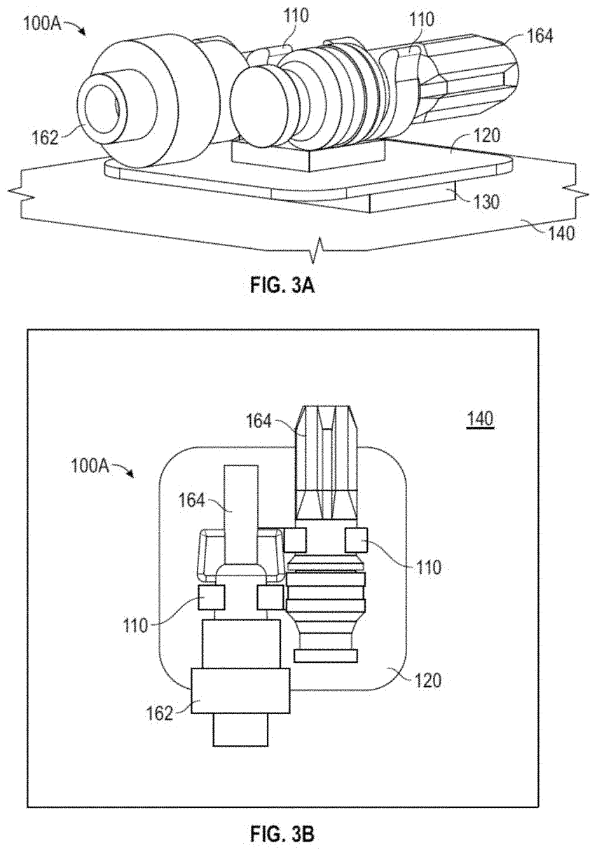

FIGS. 3A and 3B illustrate another embodiment of the securement device 100A. Like reference numerals denote like features that have been described in the previous sections. The only difference between securement device 100 (that has been previously described) and securement device 100A relates to the number of retention formations in the form of clips 110. Securement device 100A comprises two retention clips 110 (instead of three retention clips 110 shown in device 100).

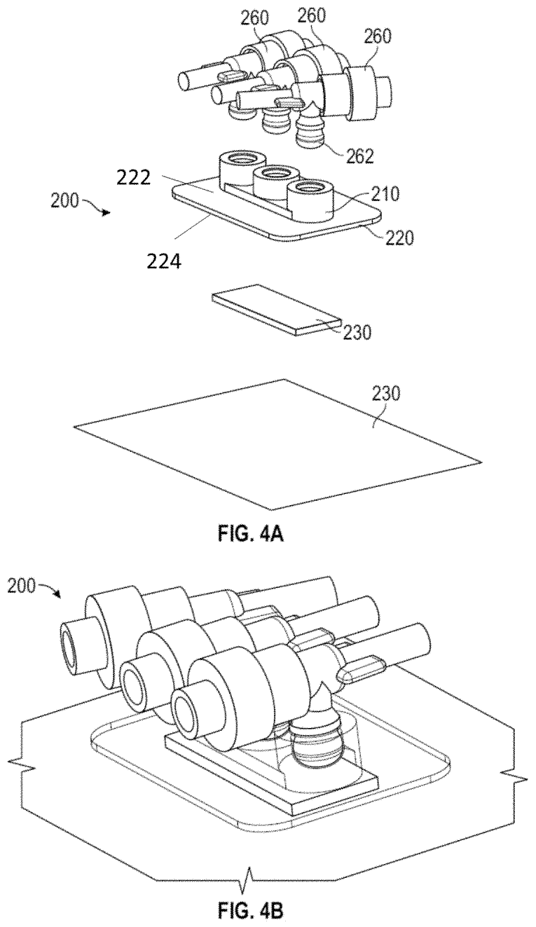

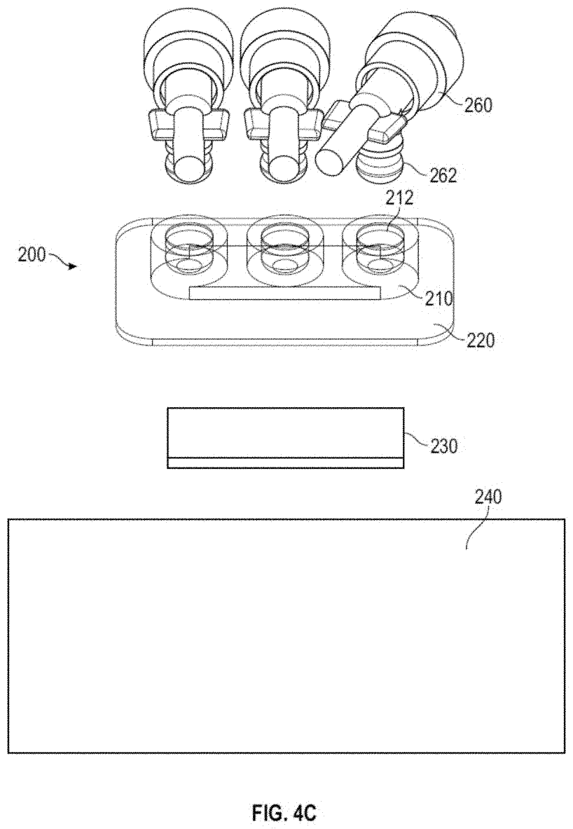

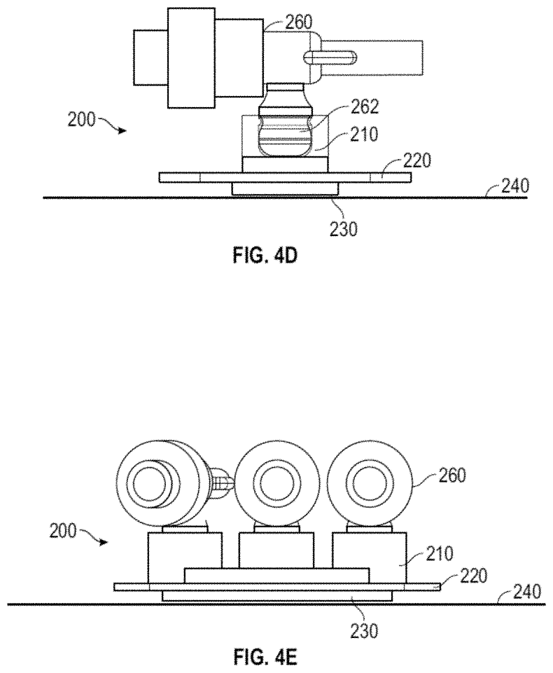

FIGS. 4A to 4E illustrate yet another embodiment of a securement device 200. The securement device 200 comprises a base 220 having a continuous top surface 222 and an underside surface 224. Once again the continuous top surface 222 may include an assembled film or coating with a specified antimicrobial additive and the underside surface 224 of the base 220 has a medical grade adhesive tape 230 positioned under it. The securement device 200 comprises a retention formations in the form of female connectors 210 that are adapted to engage a male connecting portion 262 extending outwardly and downwardly from the housing of each of the three port bodies 260. Each of the female retention formations 210 defines a substantially hollow cylindrical body with an opening 212 and the hollow cylindrical bodies project in an in-use upwardly direction from the top surface of the base 220 so that the male connecting portions 262 (which project from the outer surface of the housing forming the port bodies 260) can be press-fitted into the respective female connecting portions 210.

Unlike, the previously described embodiment, the securement device 200 requires the port bodies 260 to include a complementary configuration that enables the female connecting formation 210 to be easily coupled with the male connecting portion 262 by way of press-fitting.

The novel arrangement of the securement device 200 in combination with the unique configuration of the port bodies 260 of the catheter extension set prevents the port bodies 260 and the connected fluid lines from being inadvertently caught or pulled whilst they are connected with the catheter hub. As explained in earlier sections, the base 220 may be coated with antiseptic and/or antimicrobial agent which further reduces chances of port access infection. Once again, a chloroxylenol impregnated transparent film 240 may be placed over the user's body before adhesively bonding the base 220 to the transparent film to address the issue of central line-associated bloodstream infection (CLABSI) because the base 220 forms a barrier between the port bodies 260 and the patient's body.

It is also important to note that in at least some embodiments, the invention may encompass a kit that includes the securement device 200 either alone or in combination with the modified port bodies 260 (which includes the male connecting portion 262) and other parts of the catheter extension assembly. In yet another embodiment, the invention also relates to the port bodies 260 of the catheter connector either alone or in combination with the securement device 200.

It is to be understood that the base 220 may also include an antimicrobial layer on the top surface 222 of the base 220 particularly around the regions of the top surface where the female connectors 210 have been formed on the top surface 222 for preventing infections within the port bodies 260 and the terminal ends of the catheters connected thereto.

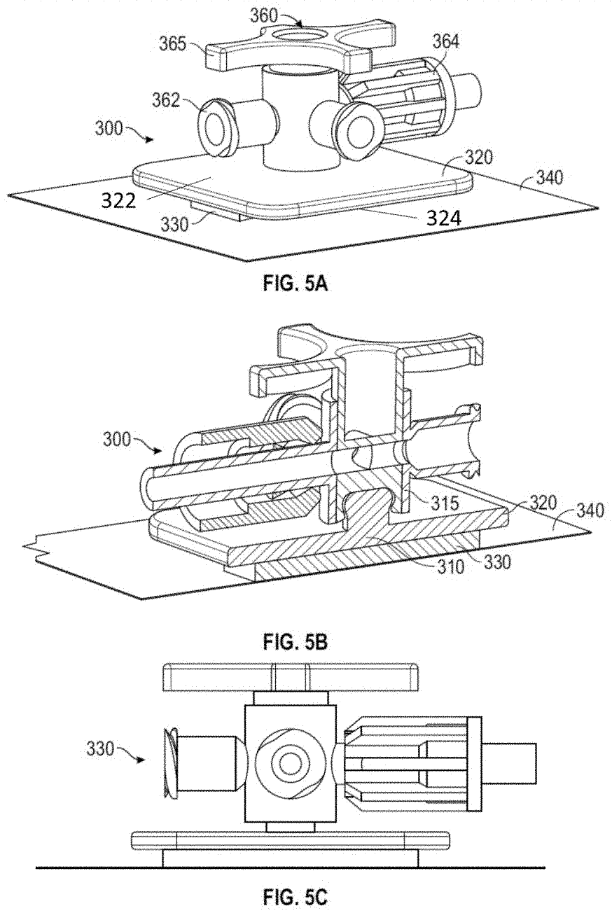

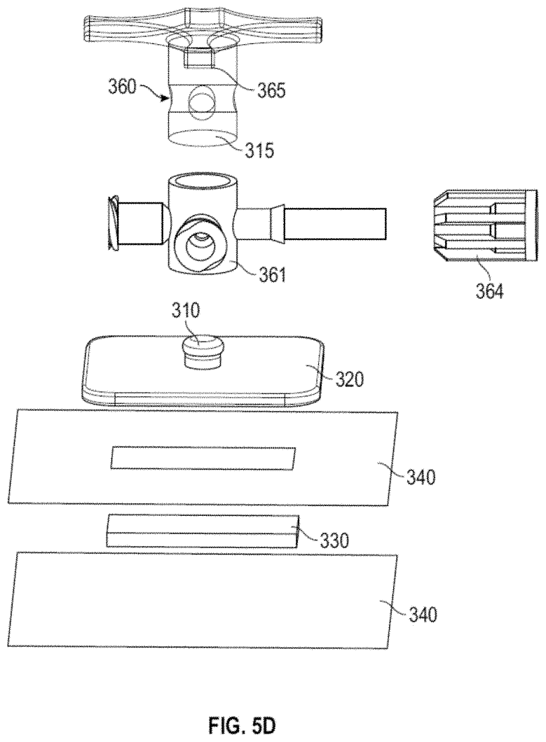

Referring to FIGS. 5A to 5D, yet another embodiment of the securement device 500 and a three way stop cock connector 360 (suitable for use in medical applications such as administration of different products, such as serum, nutrients and medications through an intravenous catheter) is shown.

The securement device 300 comprises a base 320 having a continuous top surface 322 and an underside surface 324. Once again the continuous top surface 322 may include an assembled film or coating with a specified antimicrobial additive and the underside surface 324 of the base 320 has a medical grade adhesive tape 330 positioned under it. The securement device 300 comprises a retention formations in the form of a male connecting portion 310 that is adapted to engage a female connecting portion 315 provided in an in-use lower part of the housing that forms the stop cock connector 360. The retention formation comprising the male connecting portion 310 extends upwardly from the top surface of the base 320. The female connecting portion of the stop cock 360 is located at an in-use lower portion of the knob body 365 which is movably received in an outer housing which defines the inlet ports 362 and the outlet port 364. The male connecting portion 310 is adapted to be received into the female connecting portion 315 of the stop cock housing 360. The male connecting member 310 includes a shoulder that assists with the male connecting portion 310 being press-fitted into a lower end of the housing that forms female connecting portion 315. Once again, the housing comprises a complementary configuration that allows the housing to be securely coupled with the securement device 300 by press-fitting.

The stop cock 360 includes a knob body 365 which is movably received within a sleeve with the two ports 362. The knob body 365 can be turned to select one of the two ports 362 for conveying fluid to the catheter hub. A lower portion of the knob body 365 defines the female connecting portion 315 which can be press fitted into the male connecting portion 310 of the securement device 300. The upper portion of the knob body 365 can be pulled away to uncouple the female connecting portion 315 from the male connecting portion 310.

The novel arrangement of the securement device 300 in combination with the unique configuration of the stop cock housing 360 of the catheter extension prevents the ports 362 and the connected fluid lines from being inadvertently caught or pulled whilst they are connected with the catheter hub. As explained in earlier sections, the top surface 322 of the base 320 may be coated with antiseptic and/or antimicrobial agent particularly at and adjacent the male connecting portion 310 which further reduces chances of port access infection. Once again, a chloroxylenol impregnated transparent film 340 may also be placed over the user's body before adhesively bonding the base 320 to the transparent film to address the issue of central line-associated bloodstream infection (CLABSI) because the base 320 effectively forms a barrier between the ports 362 and the patient's body.

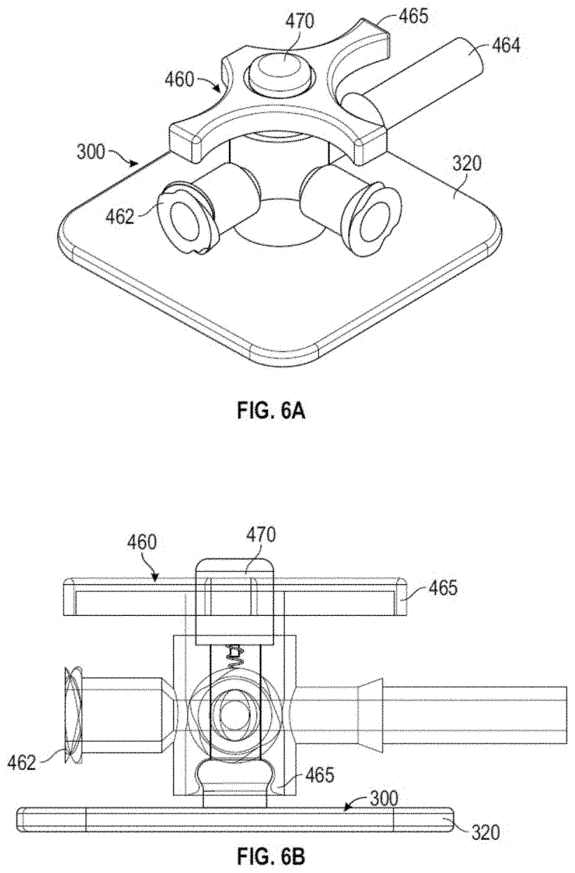

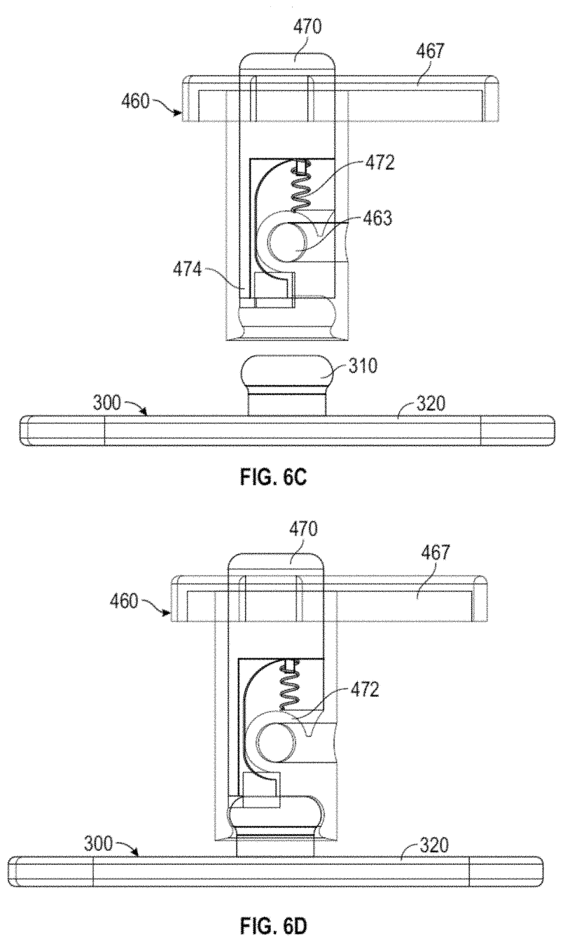

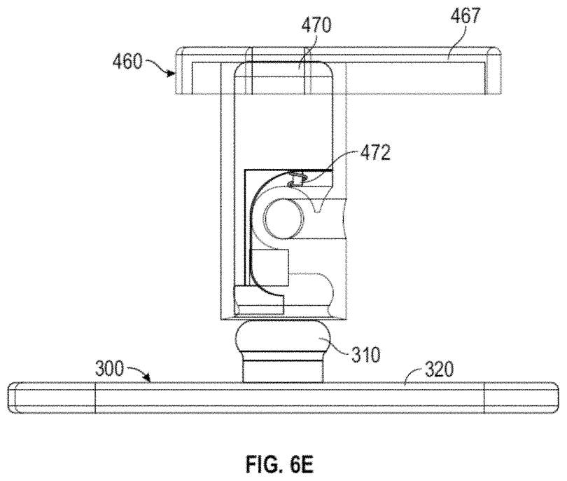

Referring to FIGS. 6A to 6E, the securement device 300 has been shown in an in-use configuration with another embodiment of the stop cock 460. Like reference numerals denote like features that have been previously described in earlier sections. The stop cock 460 includes a knob 465 that can be turned to control flow of fluid from the inlet ports 462 towards the catheter hub (not shown) via an outlet port 464. A lower portion of the knob body 465 is received into a bore of the outer hosing (that includes the inlet ports 462 and the outlet ports). The knob body includes a transversely disposed passage which when turned into an open position allows fluid to be conveyed selectively from either of the inlet ports 462 to the outlet port 464. A lower portion of the knob body 465 includes a female connecting portion 415 which can be press-fitted onto the male connecting portion 310. The knob body 465 also includes a lumen for receiving a spring loaded member 470 that can be pushed to release the female connecting portion 415 from the male connecting portion 310 of the securement device 300. An upper portion of the spring loaded member 470 can be pressed in a downwardly direction (against the bias applied by biasing spring 472) so that a lower portion 474 of the release member 470 pushes against the male connecting portion 310 of the securement device 300. A user may position their fingers below the upper tabs of the knob body 465 to simultaneously apply a pulling force which in combination with the pushing force applied on the male connecting portion 310 (due to downward pushing of the release member 470) results in the stop cock 460 becoming uncoupled from the securement device 300 (see FIG. 6E).

Once again, the novel arrangement of the securement device 300 in combination with the unique configuration of the stop cock housing 460 of the catheter extension set prevents the stop cock 460 and the connected fluid lines from being inadvertently caught or pulled whilst they are connected with the catheter hub. As explained in earlier sections, the securement device 300 has a base 320 that may be coated with antiseptic and/or antimicrobial agent on the top surface 322 which further reduces chances of port access infection. Once again, a chloroxylenol impregnated transparent film 340 may also be placed over the user's body before adhesively bonding the base 320 to the transparent film to address the issue of central line-associated bloodstream infection (CLABSI).

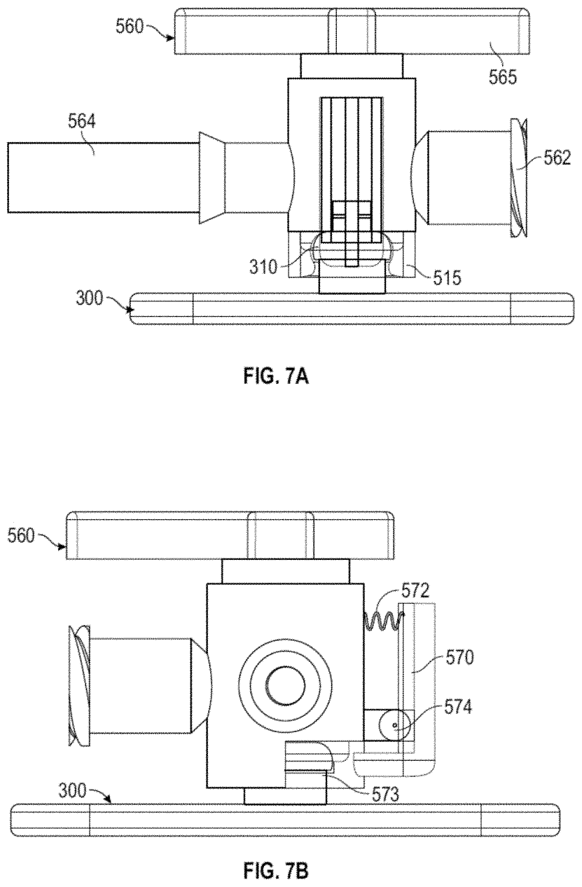

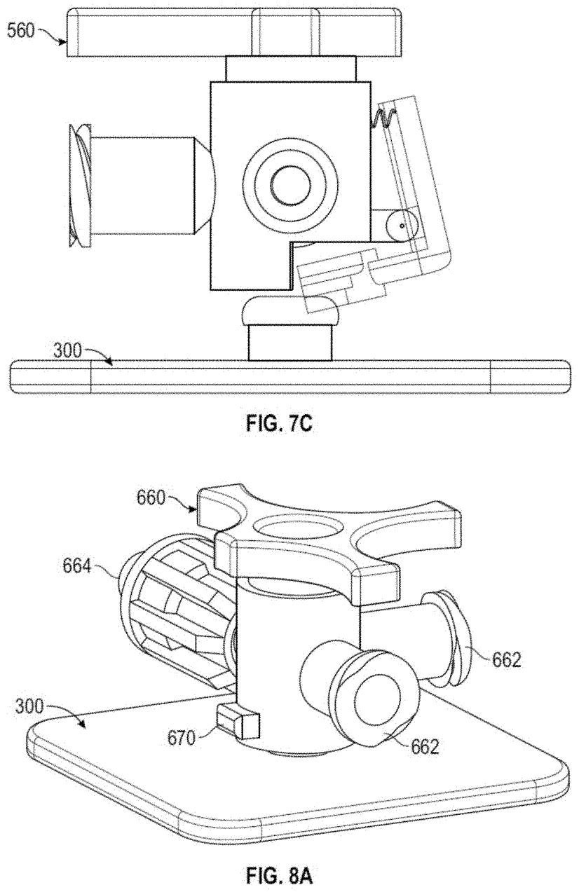

Referring to FIGS. 7A to 7D, yet another embodiment of a stop cock 560 is shown in combination with the securement device 300. Once again, like reference numerals denote like features that have been previously described in the earlier sections. The stop cock 560 includes a knob 565 that can be turned to control flow of fluid from the inlet ports 562 towards the catheter hub (not shown) via an outlet port 564. A lower portion of the knob body 565 is received into a bore of the outer hosing (that includes the inlet ports 562 and the outlet port 562). The knob body includes a transversely disposed passage which when turned into an open position allows fluid to be conveyed selectively from either of the inlet ports 562 to the outlet port 564. A lower portion of the outer body that defines the outlet port 562 and the inlet port 564 includes a female connecting portion 515 at a lower end which can be press-fitted onto the male connecting portion 310.

The stop cock assembly 560 also includes a spring loaded member lever 570 that can be turned (against the bias provided by a spring body 572) to release the female connecting portion 515 from the male connecting portion 310 of the securement device 300. The release lever 570 is pivotally coupled to an engagement portion 573 which is positioned in between a lower portion of the outer housing of the stop cock 560 (which defines the inlet ports 562 and the outlet port 564) and the male connecting portion 310 of securement device 300. Movement of the 570 lever in a first direction (against the biasing force applied by the biasing spring 5720) results in the female connecting portion 515 of the outer housing becoming uncoupled from the male connecting portion 310 of the securement device 300.

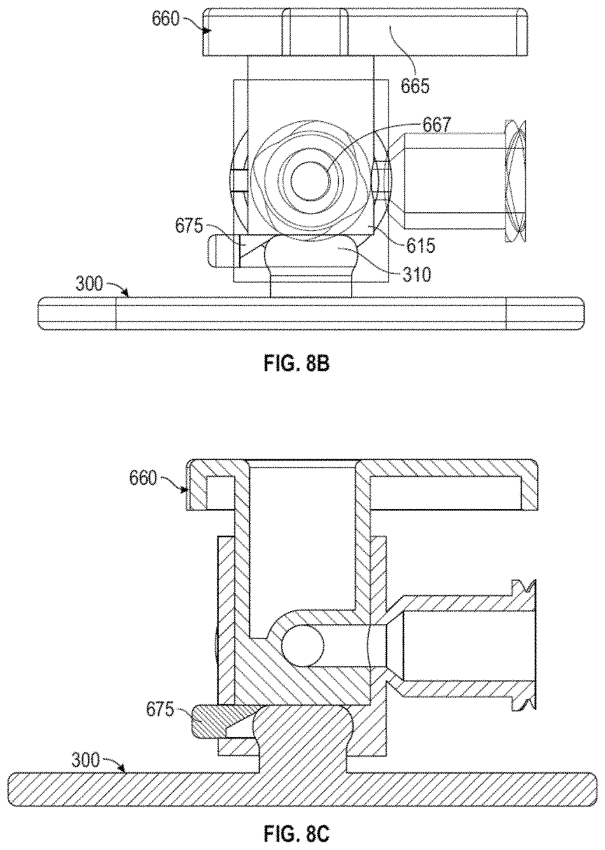

Referring to FIGS. 8A to 8C yet another embodiment of the stop cock 660 is shown in combination with the securement device 300. Once again, like reference numerals denote like features that have been previously described in the earlier sections. The stop cock 660 includes a knob 665 that can be turned to control flow of fluid from the inlet ports 662 towards the catheter hub (not shown) via an outlet port 664. A lower portion of the knob body 665 is received into a bore of the outer hosing (that includes the inlet ports 662 and the outlet port 664). The knob body 665 includes a transversely disposed passage 667 which when turned into an open position allows fluid to be conveyed selectively from either of the inlet ports 662 to the outlet port 664. A lower portion of the outer body that defines the outlet port 662 and the inlet port 664 includes a female connecting portion 615 at a lower end which can be press-fitted onto the male connecting portion 310 of the securement device 300.

The stop cock assembly 660 also includes a release member 670 that can be pushed to release the female connecting portion 615 from the male connecting portion 310 of the securement device 300. The release member comprises an inner wedge portion 675 that when pushed positions the wedged portion 675 in between the male connecting portion 310 and the female connecting portion 615 located at the lower end of the knob body 665 thereby allowing the stop cock 660 becoming uncoupled from the securement device 300.

In compliance with the statute, the invention has been described in language more or less specific to structural or methodical features. The term “comprises” and its variations, such as “comprising” and “comprised of” is used throughout in an inclusive sense and not to the exclusion of any additional features.

It is to be understood that the invention is not limited to specific features shown or described since the means herein described comprises preferred forms of putting the invention into effect.

The invention is, therefore, claimed in any of its forms or modifications within the proper scope of the appended claims appropriately interpreted by those skilled in the art.

What is claimed is:

1. A securement device for securement of a catheter connecting assembly to a body of a patient wherein the catheter connecting assembly includes two or more ports adapted to be coupled with the catheter, each port being formed by a respective port body wherein the ports are adapted to be fluidly coupled to a catheter hub of a catheter, the securement device comprising:
a base having a continuous top surface and a continuous bottom surface wherein at least a portion of the top surface comprises an antimicrobial layer and wherein at least a portion of the bottom surface comprises an adhesive for attachment of the base to the body of the patient and wherein the base is dimensioned to prevent direct contact between said ports and body of the patient; and
one or more retention formations located on the top surface of the base for receiving and retaining at least a portion of a housing comprising said one or more port bodies adapted to be coupled with a terminal end of the catheter wherein the retention formations are structured to prevent direct contact between the top surface of the base and the port bodies and wherein said retention formations are located on said portion of the top surface comprising the antimicrobial layer.

2. (canceled)

3. (canceled)

4. (canceled)

5. A securement device in accordance with claim 1 wherein the retention formations project in an in-use upwardly direction to receive and retain at least a portion of a housing defining said one or more port bodies.

6. A securement device in accordance with claim 1 wherein the antimicrobial layer on the top surface of the base is an assembled film or a substrate coated with one or more antimicrobial additives.

7. A securement device in accordance with claim 1 further comprising a removable covering to cover said portions of the top surface with the antimicrobial coating.

8. (canceled)

9. A securement device in accordance with claim 1 wherein the retention formations are structured to allow said at least portions of the housing defining the one or more port bodies to be press-fitted or snap-fitted thereon by way of pushing the housing in a generally downwardly direction into the top surface of the base.

10. A securement device in accordance with claim 1 wherein the retention formations comprise a plurality of clips, each clip being configured to allow a housing portion of the body to be snap fitted into the clip.

11. A securement device in accordance with claim 6 wherein each clip comprises a medial portion defining a receiving portion positioned in between biased arms that define an opening to receive the housing of the port body into the receiving portion and wherein each of the biased arms includes an outwardly positioned guiding surface to direct the housing of a port body of the catheter connecting assembly towards the receiving portion.

12. (canceled)

13. A securement device in accordance with claim 11 wherein the medial portion of the clip member is adapted to receive and retain the housing of a port body of the catheter connecting assembly to position the passage defined by the port body to extend in a substantially parallel direction relative to the top surface of the base.

14. (canceled)

15. (canceled)

16. (canceled)

17. (canceled)

18. (canceled)

19. A securement device in accordance with claim 1 wherein the retention formation comprises a male connecting portion extending upwardly from the top surface of the base, the male connecting portion being adapted for receiving a female connecting portion of the housing defining said one or more port bodies.

20. (canceled)

21. A securement assembly comprising a catheter connecting assembly with said one or more port bodies and the securement device in accordance with claim 19 wherein the housing further comprises a releasing member for uncoupling the female connecting portion from the male connecting portion of the securement device.

22. (canceled)

23. A securement assembly in accordance with claim 21 wherein the releasing member is movably positioned within a bore of the housing, the releasing member having an in-use upper end portion and an in-use lower end portion whereby pushing the releasing member in a generally downward direction results in the lower end portion of the releasing member being pushed against an upper portion of the male connecting portion and wherein a biasing arrangement being arranged to apply a bias against the releasing member being pushed in said generally downward direction.

24. (canceled)

25. A securement assembly in accordance with claim 23 wherein the housing further comprises one or more inlet ports and one or more outlet ports, said inlet and outlet ports being provided along outer walls of the housing with a flow passage extending between said inlet and outlet ports in a transverse direction relative to the bore of the housing with a controller positioned relative to the housing for controlling flow of fluid from the inlet port to the outlet port.

26. A securement assembly in accordance with claim 25 wherein the release member comprises a cavity positioned between the upper end and the lower end of the release member, the cavity being dimensioned for accommodating the flow passage to be positioned therein during use.

27. A securement assembly in accordance with claim 26 wherein the release member comprises a constricted middle portion to define said cavity, the middle portion being dimensioned to be positioned between an inner wall of the housing and the walls of the flow passage extending across the internal volume of the housing.

28. (canceled)

29. A securement assembly in accordance with claim 21 wherein an in-use upper portion of the housing comprises a tab member for receiving a user's fingers to allow a user to simultaneously push down the releasing member in a downward direction and pull the housing in an upward direction to uncouple the female connecting portion of the housing from the male connecting portion of the securement device.

30. (canceled)

31. (canceled)

32. (canceled)

33. (canceled)
